SEQUENCE LISTING (10 sequences)

SEQ ID NO: 1 - DNA, 21 bp, Mus musculus
  primer_bind (1)..(1): TNF alfa forward
agcccacgtcgtagcaaacca

SEQ ID NO: 2 - DNA, 20 bp, Mus musculus
  primer_bind (1)..(1): TNF alfa reverse
gcaggggctcttgacggcag

SEQ ID NO: 3 - DNA, 25 bp, Mus musculus
  primer_bind (1)..(1): IL-6 forward
cctctctgcaagagacttccatcca

SEQ ID NO: 4 - DNA, 22 bp, Mus musculus
  primer_bind (1)..(1): IL-6 reverse
agcctccgacttgtgaagtggt

SEQ ID NO: 5 - DNA, 20 bp, Mus musculus
  primer_bind (1)..(1): MCP-1 forward
ccagcaccagcaccagccaa

SEQ ID NO: 6 - DNA, 22 bp, Mus musculus
  primer_bind (1)..(1): MCP-1 reverse
tggggcgttaactgcatctggc

SEQ ID NO: 7 - DNA, 22 bp, Mus musculus
  primer_bind (1)..(1): IL-4 forward
aggtcacaggagaagggacgcc

SEQ ID NO: 8 - DNA, 21 bp, Mus musculus
  primer_bind (1)..(1): IL-4 reverse
tgcgaagcaccttggaagccc

SEQ ID NO: 9 - DNA, 24 bp, Mus musculus
  primer_bind (1)..(1): IFN gamma
gccaagtttgaggtcaacaaccca

SEQ ID NO: 10 - DNA, 20 bp, Mus musculus
  primer_bind (1)..(1): IFN gamma reverse
cccaccccgaatcagcagcg

(19) United States
(12) Patent Application Publication
(10) Pub. No.: US 2014/0179773 A1
(43) Pub. Date: Jun. 26, 2014

(54) METHOD FOR THE TREATMENT OF HEART RELATED DISORDERS IN NAFLD PATIENTS

(71) Applicant: INSTITUTO BIOCHIMICO ITALIANO GIOVANNI LORENZINI S.P.A., Aprilia (IT)

(72) Inventor: Federico Salamone, Catania (IT)

(73) Assignee: Instituto Biochimico Italiano Giovanni Lorenzini S.p.A., Aprilia (IT)

(21) Appl. No.: 13/721,568

(22) Filed: Dec. 20, 2012

(51) Int. Cl. A61K 31/357

(52) U.S. Cl. CPC A61K 31/357
USPC 514/452

(57) ABSTRACT

The present invention concerns the field of heart related disorders in NAFLD patients, and in particular relates to a method of treatment suitable for improving the conditions of heart injury and myocardial related damage in such patients.

Cardiovascular diseases (CVD), including coronary heart disease and non-ischemic cardiomyopathy, are the leading cause of death in patients with NAFLD Silibinin administration markedly demonstrated an effectiveness on myocardial damage and an improvement of the myocardiocyte function reversing the accumulation of lipid droplets and consequently improving the condition of these patients.

### FIELD OF THE INVENTION

The present invention concerns the field of heart related disorders in NAFLD patients, and in particular relates to a method of treatment suitable for improving the conditions of heart injury and myocardial related damage in such patients.

### STATE OF THE ART

Nonalcoholic fatty liver disease (NAFLD) is a chronic metabolic disorder with significant impact on cardiovascular and liver mortality[1]. NAFLD includes a wide spectrum of lesions ranging from nonalcoholic fatty liver (NAFL) to nonalcoholic steatohepatitis (NASH) [2]. While NAFL is not generally considered a progressive liver disease, NASH may have a cirrhotic and tumorigenic evolution, causing liver-related morbidity and mortality [1]. Nevertheless, cardiovascular diseases (CVD), including coronary heart disease and non-ischemic cardiomyopathy, are the leading cause of death in patients with NAFLD [1].

The pathophysiological hallmark of NAFLD is insulin resistance (IR), and the increase in intrahepatic triglycerides (IHTG) is directly related to the impairment of insulin action in the liver, skeletal muscle, and adipose tissue of obese subjects [3-5]; recently, the Framingham Heart Study has shown that IHTG content predicts the glucose and lipid abnormalities of the metabolic syndrome independently of visceral fat [6,7]. Furthermore, liver fat content is an independent indicator of myocardial IR and impaired coronary functional capacity in diabetic patients [8], thus suggesting that NAFLD is not merely a marker, but may be actively involved in the onset and progression of CVD. On the other hand, myocardial triglyceride content is directly related to the degree of heart dysfunction both in human and rodent models [9]. Myocardial fat causes alterations in substrate utilization (cardiac work/myocardial oxygen consumption) that occur early in the cascade of events leading to impaired ventricular contractility [9,10]. Recently, echocardiographic features of early left ventricular dysfunction and impaired energetics, measured by cardiac 31P-magnetic resonance spectroscopy, have been reported in NAFLD patients in the absence of obesity, hypertension and diabetes [11]. NAFLD pathogenesis is related to a puzzling crosstalk between liver, muscle and adipose tissue about free fatty acids (FFA) utilization, leading to an increased supply of FFA to the liver which combined with de novo lipogenesis determines an abnormal IHTG content [12]. The increased availability of FFA in the liver promotes FFA oxidation and increases the production of free radicals leading to lipoperoxidation, DNA and protein damage, endogenous antioxidants depletion, and mitochondrial damage [13]. Oxidative-nitrosative stress further triggers the activation of inflammatory pathways [14,15]. Similarly to the liver, cardiac lipotoxicity is associated with increasing reactive oxygen species (ROS) and reactive nitrogen species (RNS) production, which leads to DNA damage and death of myocardiocytes [16,17]. In consideration of the coexistence of liver and myocardial injury in patients with NAFLD and of the shared molecular path-ways of damage, it is important to determine whether therapies aiming at improving liver histology would also be able to improve myocardial damage.

Silibinin is a polyphenolic compound contained in silymarin, a mixture of flavonolignans extracted from the seeds of milk thistle (Silybum marianum), used as hepatoprotective agent.

Potent scavenging properties have been demonstrated in vitro and in vivo, in different hepatic and non-hepatic cells [18,19]; and strong evidences for silibinin therapeutic efficacy have been reported in different types of experimental liver injury [20-22].

Until now an effective therapy which allows to improve the conditions of heart injury and myocardial related damage in NAFLD patients has not been acknowledged. The object of the present invention is therefore the development of a method of treatment of heart injury and myocardial related damage in NAFLD patients.

### SUMMARY OF THE INVENTION

The present invention concerns a method of treating heart related disorders in NAFLD patients, comprising the step of administering Silibinin to a subject in need thereof.

As will be further described in the detailed description of the invention, the method of the present invention has the advantages of being specific for improving the conditions of heart injury and myocardial related damage in such patients.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-4, wherein:

FIG. 1. Effects of silibinin on liver and heart injury. (A) Hematoxylin-eosin stained liver sections of vehicle db/m mice showed normal morphology. (B) Liver sections of db/db mice fed a methionine-choline deficient (MCD) diet revealed severe azonal steatosis, diffuse hepatocyte ballooning and scattered inflammatory foci. (C) Liver sections of silibinin-treated mice evidenced marked decrease of steatosis, reduced ballooning and absence of inflammatory cells. (D) Hematoxylin-eosin stained sections of myocardial tissue demonstrated normal appearance. (E) Myocardial sections of vehicle db/db mice fed MCD diet showed diffuse vacuolar degeneration. (F) Myocardial sections of silibinin-treated animals demonstrated absence of fat accumulation and regular myocardiocytes morphology. (G) Overall, nonalcoholic fatty liver disease (NAFLD) activity score was significantly decreased in animals treated with silibinin. (H) Myocardiocytes morphology was preserved in the silibinin group. *P<0.05 vs db/m+SD, **P<0.05 vs db/db+MCD [magnification: 10× (A-C); 40× (D-F)].

FIG. 2. Effects of silibinin on liver oxidative stress and inflammatory cytokines. (A) Isoprostanes and (B) 8-deoxyguanosine (8-OHG) were markedly increased in vehicle db/db fed a methionine-choline deficient (MCD) diet and significantly decreased by silibinin. (C) GSH levels was completely restored by silibinin administration. (D) Nitrite/nitrates were increased in vehicle db/db fed MCD diet whereas silibinin restored them to the levels of lean mice. (E) TNF-α_gene expression was reversed by silibinin treatment. (F) IL-6 was significantly decreased in vehicle db/db fed MCD diet whereas increased by silibinin administration. *P<0.05 vs db/m+SD, **P<0.05 vs db/db+MCD.

FIG. 3. Effects of silibinin on heart oxidative stress and inflammatory cytokines. (A) Isoprostanes and (B) 8-deoxyguanosine (8-OHG) were markedly increased in vehicle db/db fed a methionine-choline deficient (MCD) diet and decreased to the levels of lean controls by silibinin. (C) GSH levels were also restored by silibinin treatment. (D) Nitrite/nitrates were significantly increased in vehicle db/db fed MCD diet whereas silibinin returned them to the levels of lean mice. (E) TNF-α protein levels were strongly increased in the myocardium of vehicle db/db fed MCD diet and decreased to the levels of lean animals by silibinin. (F) IL-6 levels were decreased in the heart of vehicle db/db fed MCD diet and were restored to the levels of lean animals by silibinin administration. *P<0.05 vs db/m+SD, **P<0.05 vs db/db+MCD.

FIG. 4. Effects of silibinin on liver JNK phosphorylation. Immunohistochemistry for p-JNK was performed on liver sections from (A) vehicle db/m, (B) vehicle db/db fed a methionine-choline deficient (MCD) diet and (C) silibinin-treated db/db fed MCD diet. In agreement with the improvement of insulin resistance, redox and inflammatory status we observed a decrease of immune-positivity for phosphorylated JNK in silibinin-treated vs vehicle db/db mice fed MCD diet.

### DETAILED DESCRIPTION OF THE INVENTION

The present invention concerns a method of treating heart related disorders in NAFLD patients, comprising the step of administering Silibinin to a subject in need thereof.

Heart disorders are quite common and vary from infections of heart valves to fat accumulation in the coronary vessels. The most common medical disorder to affect the heart is coronary artery disease. Cardiovascular diseases such as coronary disorders and non-ischemic cardiomyopathy are the leading cause of death in patients with NAFLD.

When fat accumulates in tiny vessels that supply to the oxygen to the heart, blood supply to the heart can be compromised. When the heart fails to receive blood, it causes pain which is medically known as angina.

In a further aspect, the invention provides a method of treating heart related disorders in NAFLD patients, wherein the heart related disorders are selected from the group consisting of hypertension, congestive heart failure, heart injury, coronary artery disease, heart oxidative stress, myocardial injury, myocardial infarction, non-ischemic cardiomyopathy and myocardial related damage.

In a preferred aspect the heart related disorder is selected from the group consisting of myocardial injury, myocardial infarction and myocardial related damage still more preferably myocardial injury. Myocardial fat causes alterations in substrate utilization (cardiac work/myocardial oxygen consumption) that occur early in the cascade of events leading to impaired ventricular contractility.

In a further aspect, the invention provides a method of treating heart related disorders in NAFLD patients, wherein after the treatment, the morphological abnormalities of the myocardiocytes of the patient are reversed and the myocardial morphology is similar to that of a healthy individual.

The morphological abnormalities of the myocardiocytes are selected from the group consisting of diffuse vacuolar degeneration, intracellular lipid accumulation, myocardiocyte abnormal size and altered nuclear morphology.

In some patients the severity of cell damage might induce morphological/functional abnormalities and even progression of the disease to cardiac dilation and dysfunction.

In a preferred aspect, the invention provides a method of treating heart related disorders in NAFLD patients, wherein after the treatment, there is an improvement of insulin resistance, reduction of oxidative stress and restore of inflammatory signaling in the myocardiocytes.

The invention provides a method of treating heart related disorders in NAFLD patients, wherein after the treatment, there is an antisteatotic effect of silibinin in the heart.

In a further aspect, the invention provides a method of treating heart related disorders in NAFLD patients, wherein the silibinin is administered in a pharmaceutical formulation.

We explored the effect of a 4-week daily administration of silibinin both in the liver and in the myocardium of db/db mice fed a MCD diet. This animal model displayed histological features of progressive NAFLD and accumulation of lipid droplets in the myocardium, in keeping with analogous findings of myocardial steatosis in patients with obesity and diabetes [9]. The antisteatotic effect of silibinin in the heart was particularly impressive because it was largely unexpected and completely reversed myocardial damage. This study demonstrates the effectiveness on myocardial damage of a compound used for liver protection in NASH. The initiation and perpetuation of cell injury in NAFLD is associated with the increase of free radicals and the depletion of endogenous anti-oxidant defense both in human and rodents [26,27]. Several data suggest that lipotoxicity plays a crucial role also in the pathogenesis of cardiomyopathy underlying non-ischemic chronic heart failure, a leading cause of death in patients with obesity and/or diabetes [9,16]. In this NAFLD model, we observed a parallel fat accumulation in the liver and heart, in association with oxidative stress. The importance of oxidative stress in NASH pathogenesis is underscored by recent findings showing the effectiveness of vitamin E in preventing liver injury progression in patients [28]. In our animal model, silibinin was able to decrease both isoprostanes, which are sensitive markers of lipoperoxidation [29], and 8-OHG, a marker of DNA damage, which is increased in NAFLD patients in relation to degree of liver injury [30] and in patients with obesity/diabetes cardiomyopathy [17]. The used dose is higher than in the commercially available oral formulations of silymarin/silibinin, but the safety for comparable dosage of silibinin has been showed both in healthy volunteers [31] and in patients with chronic liver disease [32,33]. Previous findings reported the efficacy of a silibinin/vitamin E oral formulation on surrogate serum markers of liver injury in NAFLD patients [34] and the inhibitory effect of silibinin on human hepatic stellate cells activation in vitro [35]. The antioxidant action of silibinin in our animal model was further confirmed by its effect on GSH levels. GSH levels are decreased in NASH patients [25,26] and in mice treated with a MCD diet [26]. Restoring mitochondrial GSH to normal levels by the administration of GSH precursors prevents the establishment of inflammation in the MCD diet model [36]. A marked decrease of MRC activity have been evidenced in patients with NAFLD [37], and liver mitochondrial dysfunction seems to be an early pathogenetic step that precedes fatty liver in rats [38]. Mitochondria are a target of oxidative stress, but also the main source of free radicals and the impairment in MRC activity is directly responsible for the increase in cellular ROS in a vicious cycle [13]. Our results are consistent with previous findings on MRC restoration by silibinin in a rodent model of iron overload [20] and with recent data in another model of NASH [39]. Beyond the effects of silibinin on oxidative stress in hepatic and myocardial tissue, silibinin also displayed a significant effect on inflammatory cytokines levels. The cellular redox status is one of the main stimuli for TNF-α mediated inflammation [40]. Interestingly, we observed a stronger increase of TNF-α levels in the heart, which indicates that myocardial tissue is a main target of inflammation at the early stage of the natural history in this NAFLD model. Noteworthy, TNF-α and oxidative stress plays a synergic role for the progression of heart failure [41] and the vicious cycle inflammation-oxidative stress represents a main target for preventing myocardial damage [41]. Consistently with the observed improvement of mitochondrial function [42], silibinin administration decreased also TNF-α gene expression in the liver. Surprisingly, IL-6 expression was decreased in the liver and in the myocardium of db/db mice fed MCD diet. However, numerous experimental and clinical evidences suggest that IL-6 is anti-inflammatory and anti-atherogenic cytokine [43], i.e. IL-6 increases in some pathological conditions as a compensatory pathway. In the liver, IL-6 administration in db/db mice and in mice fed HFD decreases fatty liver and insulin resistance [44] and ameliorates mitochondria lipid disturbance in hepatocytes isolated from steatotic animals fed a choline deficient diet [45]. In the heart, it has been demonstrated that animal lacking IL-6 display an accumulation of lipids, particularly FFA and ceramides [46], and therefore it is conceivable that the decrease of IL-6 in the heart and liver of db/db mice fed a MCD diet significantly contributes to lipotoxicity. MCP-1 levels were unchanged, confirming that this cytokine does not play a relevant role in liver steatosis and inflammation induced by MCD diet [47], whereas phosphorylation of JNK, which is associated with insulin resistance and inflammation [48], was significantly decreased. In summary, this study suggests a combined effectiveness of silibinin on preventing hepatic and myocardial injury in experimental NAFLD. These effects are mediated by improvement of insulin resistance, reduction of oxidative stress, and restore of inflammatory signaling, key events in the pathogenesis of NASH. Our findings provide a rationale for clinical studies on the use of silibinin in the management of liver and cardiovascular damage in patients with NAFLD.

In a still further aspect, the invention provides a method of treating heart related disorders in NAFLD patients, wherein the silibinin is administered via the enteral or parenteral route.

Administration through the gastrointestinal tract is sometimes termed enteral or enteric administration and includes oral and rectal administration, in the sense that these are taken up by the intestines. Some application locations often classified as enteral, such as sublingual and sublabial or buccal (between the cheek and gums/gingiva), are taken up in the proximal part of the gastrointestinal tract without reaching the intestines. Strictly enteral administration (directly into the intestines) can be used for systemic administration, as well as local (sometimes termed topical). However, in the classification system basically distinguishing substances by location of their effects, the term enteral is reserved for substances with systemic effects.

### EXAMPLES

### Example 1

Silibinin Decreases Insulin Resistance and Serum ALT [Alanine Aminotransferase]

This study is aimed at assessing the efficacy of silibinin both on liver and heart injury in NAFLD and at identifying the related molecular events. In order to investigate this issue, we used db/db mice fed a methionine-choline deficient (MCD) diet, a model combining the features of the metabolic syndrome with the histological pattern of NASH [23,24]. In fact, db/db mice fed an MCD diet do partially conserve increased visceral adiposity and an insulin resistant phenotype, while developing hepatocellular injury [24].

All db/db mice weighted more than their lean controls at week 8, before starting the MCD diet (Table 1). After 4 weeks of MCD diet, at week 12, body weight was still higher in vehicle-treated animals; silibinin treatment did not significantly decrease body weight (Table 1) and did not modify food intake (data not shown). Vehicle db/db fed MCD diet had higher serum ALT when compared to lean animals; in silibinin-treated mice, ALT levels were 3-fold decreased (Table 1). Untreated db/db fed MCD diet were insulin resistant, as shown by HOMA-IR; silibinin decreased fasting glucose and insulin, completely reversing insulin resistance (Table 1).

TABLE 1
Biometric and biochemical parameters in the three experimental groups

| | | | db/db + MCD + |
|---|---|---|---|
| | db/m + SD | db/db + MCD | silibinin |
| 8-Weeks weight (g) | 24.5 ± 0.5* | 38.0 ± 2.2* | 37.5 ± 2.7* |
| 12-Weeks weight (g) | 26.8 ± 1.2 | 36.4 ± 1.8* | 34.8 ± 1.9 |
| Serum ALT (IU/L) | 38.4 ± 12.0 | 454.2 ± 80.4* | 180.5 ± 66.5*, ** |
| Blood glucose (mg/dL) | 68.2 ± 7.6 | 181.8 ± 40.2* | 103.7 ± 16.8*, ** |
| Serum insulin (mU/mL) | 6.8 ± 1.2 | 8.3 ± 0.9* | 6.5 ± 1.1** |
| HOMA-IR | 1.1 ± 0.2 | 3.7 ± 0.7* | 1.7 ± 0.5*, ** |
| Liver triglycerides | 3.8 ± 0.8 | 13.6 ± 1.3* | 8.3 ± 2.4*, ** |
| (mg/g protein) | | | |
| Liver MRC complex I | 100 ± 5.4 | 56.4 ± 4.6* | 90.2 ± 5.2** |
| Liver MRC complex II | 100 ± 4.8 | 52.2 ± 4.8* | 88.4 ± 4.3** |
| Liver MRC complex III | 100 ± 3.2 | 54.4 ± 3.6* | 94.4 ± 6.8** |
| Liver MRC complex IV | 100 ± 4.8 | 56.8 ± 4.2* | 96.1 ± 5.2** |
| Liver MRC complex V | 100 ± 5.2 | 58.6 ± 3.3* | 94.3 ± 6.2** |

SD, standard diet;
MCD, methionine-choline deficient;
MRC, mitochondrial; respiratory chain.
*P <0.05 vs db/m + SD**
P <0.05 vs db/db + MCD.

### Example 2

Silibinin Improves Hepatic and Myocardial Injury

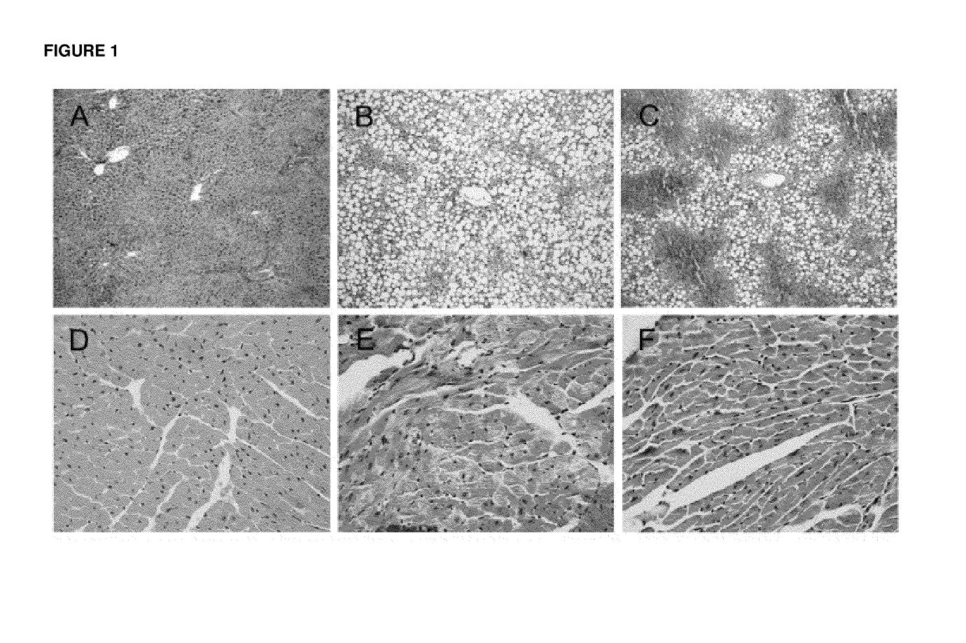

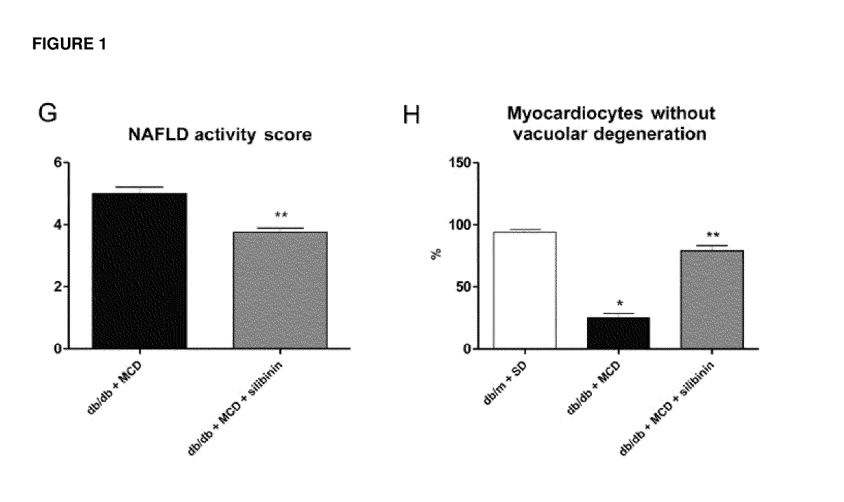

As expected, none of the lean mice presented histological features of NAFLD (FIG. 1). Liver sections of vehicle db/db fed MCD diet mice showed marked steatosis with an azonal pattern (FIG. 1), mild lobular inflammation and diffuse hepatocytes ballooning. In silibinin-treated animals steatosis was markedly reduced in grade (FIG. 1) and had a prevalent zone 3 pattern. Biochemical analysis confirmed that silibinin induced a marked decrease of liver triglycerides content in db/db fed MCD diet (Table 1). Ballooning degeneration was observed in the liver sections of all db/db fed MCD diet but was less pronounced in those treated with silibinin. Moreover, lobular inflammation was almost absent in silibinin-treated mice. Overall, NAS was significantly decreased in silibinin-treated animals (FIG. 1). Similarly, the myocardium of db/db fed MCD diet showed diffuse vacuolar degeneration at hematoxylin-eosin staining, consistent with intracellular accumulation of lipids (FIG. 1); myocardiocytes with abnormal size and altered nuclear morphology were also observed. Consistently with liver findings, silibinin treatment markedly improved myocardial injury and reversed the morphological abnormalities in most myocardiocytes (FIG. 1).

### Example 3

Silibinin Counteracts Liver and Heart Oxidative Stress and Inflammation

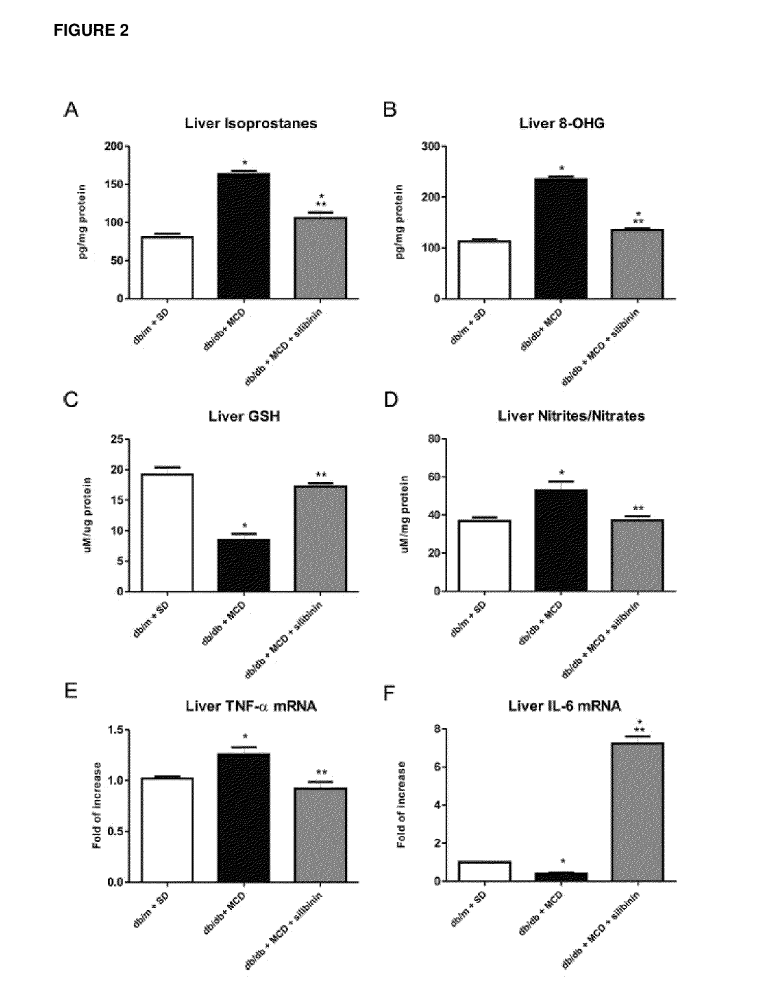

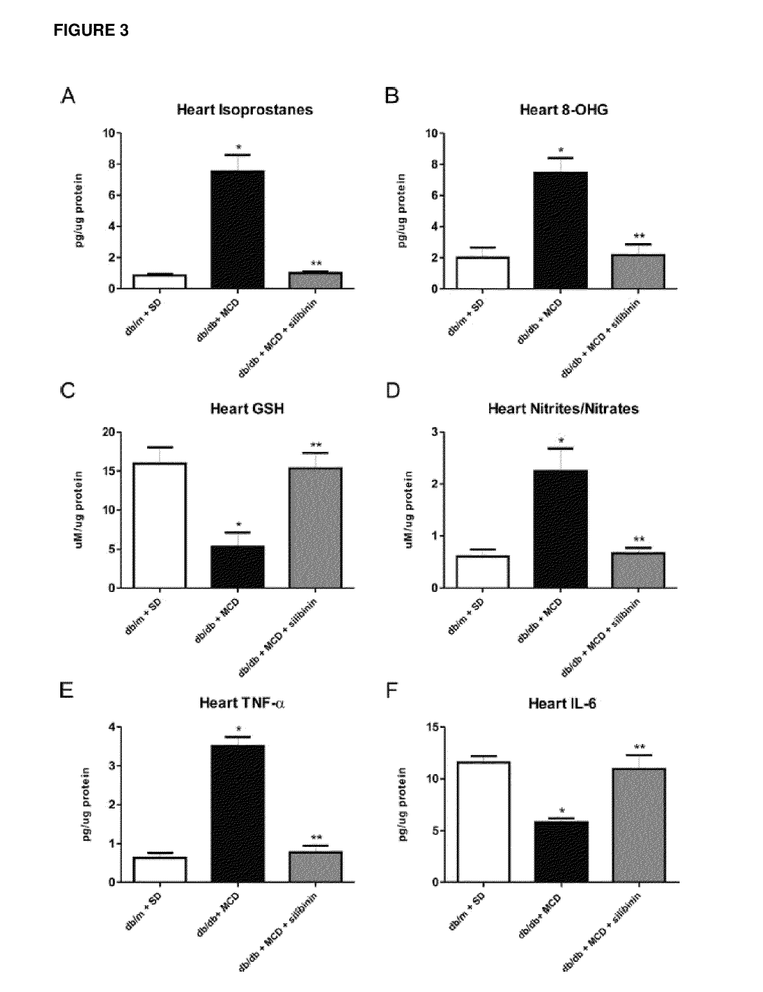

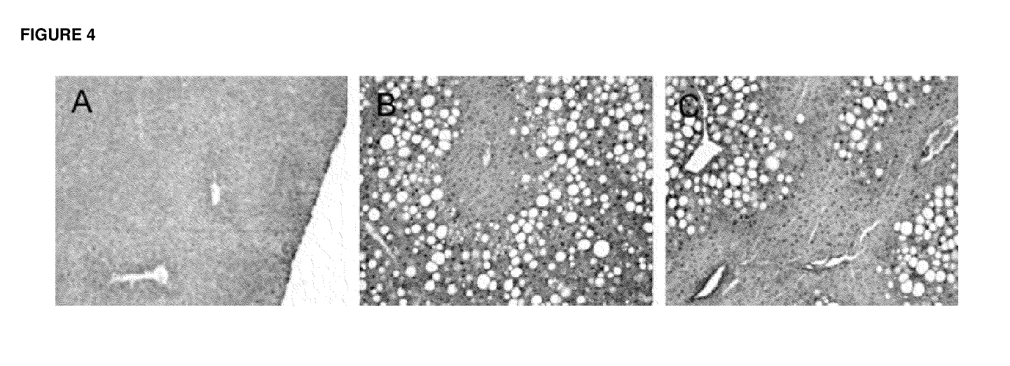

In comparison to lean controls, isoprostanes and 8-OHG, markers of lipoperoxidation and DNA damage, were markedly increased both in liver and heart of vehicle db/db fed MCD diet (FIGS. 2 and 3). Silibinin treatment significantly decreased liver isoprostanes and 8-OHG (FIG. 2); strikingly, heart isoprostanes and 8-OHG were decreased to the levels of lean controls (FIG. 3). The main scavenger GSH was decreased by 50% both in livers and hearts of vehicle db/db fed MCD diet as compared to control animals, whereas treatment with silibinin restored GSH content to lean mice levels (FIGS. 2 and 3). Likewise oxidative stress, also nitrosative stress, as assessed by nitrite/nitrates levels, was significantly increased in the liver and heart of db/db fed MCD diet and was restored to lean controls levels by silibinin (FIGS. 2 and 3). Consistently, the activity of the five complexes of MRC was reduced by 50% in vehicle db/db fed MCD diet and was completely restored by silibinin administration (Table 1). Gene expression of TNF-α was slightly increased in the liver of db/db fed MCD diet as compared with lean animals, whereas IL-6 was reduced; silibinin administration reversed gene expression of both (FIG. 2). MCP-1, IL-4 and IFN gene expression was not significantly modified in any group (data not shown). Similarly, heart TNF-α protein expression was increased and IL-6 markedly decreased in the vehicle group. Silibinin was able to restore heart TNF-α and IL-6 to lean animals levels (FIG. 3). In agreement with the improvement of insulin resistance and of redox and inflammatory status, we observed a decrease of immunohistochemical staining for phosphorylated JNK isoforms in MCD fed db/db mice treated with silibinin versus mice only receiving the steatogenic diet (FIG. 4).

Materials and Methods

Animals and Treatments

All procedures fulfilled the Italian Guidelines for the Use and Care of Laboratory Animals. Six-week-old male BKS.Cg-m+/+ Leprdb/J (db/db) obese mice and six-week-old male heterozygous db/m lean control mice were purchased. Animals were maintained in a temperature and light-controlled facility and permitted ad libitum consumption of water; after two week of acclimation, db/db mice were fed a MCD diet for 4 weeks, whereas db/m mice were fed a MCD diet supplemented with methionine and choline, i.e. a standard diet (SD), for the same period. Silibinin was dissolved in saline (vehicle) and daily administered intraperitoneally at a dosage of 20 mg/kg of body weight. Mice were distributed in 3 groups: group I included 6 db/m mice fed a control diet and treated with vehicle (db/m+SD); group II comprised 6 db/db mice fed a MCD diet and treated with vehicle (db/db+MCD); group III included 6 db/db mice fed a MCD diet and treated with silibinin (db/db+MCD+silibinin). Treatment was administered for a 4-week period; at the end of it, animals were sacrificed after an overnight fast. Blood, liver and heart samples were obtained and stored at −80° C. for further analysis.

Histopathology and Immunohistochemistry

For conventional histopathological evaluation formalin-fixed paraffin-embedded liver and heart sections were stained with hematoxylin-eosin, using standard procedures. Liver injury was blindly evaluated according to the NAFLD activity score (NAS). Moreover, in order to investigate even modest deposition of extracellular matrix components Sirius Red staining was performed on formalin-fixed paraffin-embedded liver sections as previously described [25] with a single modification: briefly, after the usual steps to stain liver sections (2 μm thick) in 0.1% Sirius Red F3B in a picric acid solution (1.2%), sections were further rapidly exposed to Harry's hematoxylin (2 s) in order to stain nuclei. Immunohistochemistry was performed on formalin-fixed paraffin-embedded liver sections. Sections (2 μm thick) were incubated with the primary antibody or anti-Thr183/Tyr185 phosphorylated-JNK, final dilution 1:50. Briefly, after microwave anti-gen retrieval, primary antibodies were labeled by using EnVision, HRP-labelled System (DAKO) antibodies directed against mouse antigen and visualized by 3-diaminobenzidine substrate. Negative controls were performed by replacing the respective primary antibodies by isotype and concentrations matched irrelevant antibody.

Biochemical Analyses

Blood glucose was measured by Accuchek (Roche Diagnostics, Milan, Italy). Serum ALT and serum insulin were determined using a multichannel autoanalyzer. Liver triglycerides content was measured using a serum/tissue triglyceride colorimetric kit (Biovision, Mountain View, Calif.). Liver and heart isoprostanes and 8-hydroxyguanosine (8-OHG) were determined by enzyme-linked immunosorbent assay (ELISA) test; reduced glutathione (GSH) was assessed by a GSH assay. Liver and heart nitrite/nitrates were measured colorimetrically using Griess reagent, following manufacturer's instructions. ELISA kit for TNF-α was used on whole myocardial tissue protein extracts, following manufacturer's instructions.

Mitochondrial Respiratory Chain Activity Assay

Mitochondrial respiratory chain (MRC) activities was determined as previously described [24]. Briefly, liver tissues (50-70 mg) were homogenized with 15 vol of 20 mmol/L KP buffer, pH 7.4, and centrifuged at 800×g for 10 min. Respiratory chain enzymes and citrate synthase activities were measured in a DU-650 spectrophotometer. Incubation temperatures were 30° C. for complexes I, II, III, V, and citrate synthase, and 38° C. for complex IV. Enzyme activities were assessed in supernatants, expressed as nanomoles of substrate used per minute per milligram of protein and referred as a percentage of the specific activity of citrate synthase, to adjust for the hepatic content of mitochondria.

RNA Extraction and Real Time PCR

Total RNA was extracted by homogenizing snap frozen liver samples in TRIzol reagent. Quantitative real-time PCR was performed in 7900HT Fast Real-Time PCR System using the EXPRESS SYBRR GreenER™ qPCR SuperMix with Premixed ROX (Invitrogen).

The following primers sequences were used:

| | |
|---|---|
| TNF-α fwd | 5′-AGCCCA-CGTCGTAGCAAACCA-3′, |
| TNF-α rev | 5′-GCAGGGGCTCTTGACGGCAG-3′; |
| IL-6 fwd | 5′-CCTCTCTGCAAGAGACTTCCATCCA-3′, |
| IL-6 rev | 5′-AGCCTCC-GACTTGTGAAGTGGT-3′; |
| MCP-1 fwd | 5′-CCAGCACCAGCACCAGCC-AA-3′, |
| MCP-1 rev | 5′-TGGGGCGTTAACTGCATCTGGC-3′; |
| IL-4 fwd | 5′-AGG-TCACAGGAGAAGGGACGCC-3′, |
| IL-4 rev | 5′-TGCGAAGCACCTTGGAAG-CCC-3′; |
| IFNγ | 5′-GCCAAGTTTGAGGTCAACAACCCA-3′ , |
| IFNγ rev | 5′-CCCACCCCGAATCAGCAGCG-3′. |

Reactions were performed in a 20 mL mixture containing cDNA, specific primers of each gene and the SYBRR GreenER™ qPCR SuperMix. Amplification conditions were as follows: 50° C. for 2 min, 95° C. for 10 min, followed by 40 cycles at 95° C. for 15 s and 60° C. for 1 min. The specific PCR products were detected by the fluorescence of SYBR Green, the double stranded DNA binding dye. The relative mRNA expression level was calculated by the threshold cycle (Ct) value of each PCR product and normalized with that of GAPDH by using comparative 2ΔΔCt method.

Statistical Analysis

Statistics were aided by GraphPad Prism (GraphPad, San Diego, Calif.). All results were expressed as mean±standard error of the mean. One-way ANOVA with Bonferroni Post Hoc analysis were used for parametric data. Kruskal-Wallis was used for non-parametric data. P values less than 0.05 were considered significant.

From the above description and the above-noted examples, the advantage attained by the method described and obtained according to the present invention are apparent.

### REFERENCES

[1] Musso G, Gambino R, Cassader M, et al. Meta-analysis: natural history of nonalcoholic fatty liver disease (NAFLD) and diagnostic accuracy of non-invasive tests for liver disease severity. Ann Med 2010 [epub ahead of print].

[2] Brunt E M. Histopathology of non-alcoholic fatty liver disease. Clin Liver Dis 2009;13:533-44.

[3] Korenblat K M, Fabbrini E, Mohammed B S, et al. Liver, muscle, and adipose tissue insulin action is directly related to intrahepatic triglyceride content in obese subjects. Gastroenterology 2008;134:1369-75.

[4] Fabbrini E, Magkos F, Mohammed B S, et al. Intrahepatic fat, not visceral fat, is linked with metabolic complications of obesity. Proc Natl Acad Sci USA 2009;106:15430-5.

[5] Salamone F, Bugianesi E. Nonalcoholic fatty liver disease: the hepatic trigger of the metabolic syndrome. J Hepatol 2011;53:1146-7.

[6] Speliotes E K, Massaro J M, Hoffmann U, et al. Fatty liver is associated with dyslipidemia and dysglycemia independent of visceral fat: the Framingham Heart Study. Hepatology 2010;51:1979-87.

[7] Salamone F, Galvano F, Li Volti G. Treating fatty liver for the prevention of cardiovascular diseases. Hepatology 2010;52:1174-5.

[8] Lautamaki R, Borra R, Iozzo P, et al. Liver steatosis coexists with myocardial insulin resistance and coronary dysfunction in patients with type 2 diabetes. Am J Physiol Endocrinol Metab 2006;291:E282-90.

[9] McGavock J M, Victor R G, Unger R H, et al. Adiposity of the heart, revisited. Ann Intern Med 2006;144:517-24.

[10] Neubauer S. The failing heart—an engine out of fuel. N Engl J Med 2007;356:1140-51.

[11] Perseghin G, Lattuada G, De Cobelli F, et al. Increased mediastinal fat and impaired left ventricular energy metabolism in young men with newly found fatty liver. Hepatology 2008;47:51-8.

[12] Trauner M, Arrese M, Wagner M. Fatty liver and lipotoxicity. Biochim Biophys Acta 2010;1801:299-310.

[13] Mantena S K, King A L, Andringa K K, et al. Mitochondrial dysfunction and oxidative stress in the pathogenesis of alcohol- and obesity-induced fatty liver diseases. Free Radic Biol Med 2008;44:1259-72.

[14] Marra F. Nuclear factor-kappaB inhibition and non-alcoholic steatohepatitis: inflammation as a target for therapy. Gut 2008;57:570-2.

[15] Videla L A, Tapia G, Rodrigo R, et al. Liver NF-kappaB and AP-1 DNA binding in obese patients. Obesity (Silver Spring) 2009;17:973-9.

[16] Brindley D N, Kok B P, Kienesberger P C, et al. Shedding light on the enigma of myocardial lipotoxicity: the involvement of known and putative regulators of fatty acid storage and mobilization. Am J Physiol Endocrinol Metab 2010;298:E897-908.

[17] Mellor K M, Ritchie R H, Delbridge L M. Reactive oxygen species and insulinresistant cardiomyopathy. Clin Exp Pharmacol Physiol 2010;37:222-8.

[18] Mira L, Silva M, Manso C F. Scavenging of reactive oxygen species by silibinin dihemisuccinate. Biochem Pharmacol 1994;48:753-9.

[19] Dehmlow C, Erhard J, de Groot H. Inhibition of Kupffer cell functions as an explanation for the hepatoprotective properties of silibinin. Hepatology 1996;23:749-54.

[20] Pietrangelo A, Borella F, Casalgrandi G, et al. Antioxidant activity of silybin in vivo during long-term iron overload in rats. Gastroenterology 1995;109:1941-9.

[21] Crocenzi F A, Sanchez Pozzi E J, Pellegrino J M, et al. Beneficial effects of silymarin on estrogen-induced cholestasis in the rat: a study in vivo and in isolated hepatocyte couplets. Hepatology 2001;34:329-39.

[22] Schumann J, Prockl J, Kiemer A K, et al. Silibinin protects mice from T celldependent liver injury. J Hepatol 2003;39:333-40.

[23] Sahai A, Malladi P, Pan X, et al. Obese and diabetic db/db mice develop marked liver fibrosis in a model of nonalcoholic steatohepatitis: role of short-form leptin receptors and osteopontin. Am J Physiol Gastrointest Liver Physiol 2004;287:G1035-43.

[24] Rinella M E, Elias M S, Smolak R R, et al. Mechanisms of hepatic steatosis in mice fed a lipogenic methionine choline-deficient diet. J Lipid Res 2008;49:1068-76.

[25] Vizzutti F, Provenzano A, Galastri S, et al. Curcumin limits the fibrogenic evolution of experimental steatohepatitis. Lab Invest 2010;90:104-15.

[26] Videla L A, Rodrigo R, Orellana M, et al. Oxidative stress-related parameters in the liver of non-alcoholic fatty liver disease patients. Clin Sci (Lond) 2004;106:261-8.

[27] Serviddio G, Bellanti F, Tamborra R, et al. Uncoupling protein-2 (UCP2) induces mitochondrial proton leak and increases susceptibility of non-alcoholic steatohepatitis (NASH) liver to ischaemia-reperfusion injury. Gut 2008;57: 957-65.

[28] Sanyal A J, Chalasani N, Kowdley K V, et al. Pioglitazone, vitamin E, or placebo for nonalcoholic steatohepatitis. N Engl J Med 2010;362:1675-85.

[29] Halliwell B, Whiteman M. Measuring reactive species and oxidative damage in vivo and in cell culture: how should you do it and what do the results mean? Br J Pharmacol 2004;142:231-55.

[30] Seki S, Kitada T, Yamada T, et al. In situ detection of lipid peroxidation and oxidative DNA damage in non-alcoholic fatty liver diseases. J Hepatol 2002;37:56-62.

[31] Kidd P, Head K. A review of the bioavailability and clinical efficacy of milk thistle phytosome: a silybin-phosphatidylcholine complex (Siliphos). Altern Med Rev 2005;10:193-203.

[32] Ferenci P, Scherzer T M, Kerschner H, et al. Silibinin is a potent antiviral agent in patients with chronic hepatitis C not responding to pegylated interferon/ribavirin therapy. Gastroenterology 2008;135:1561-7.

[33] Bares J M, Berger J, Nelson J E, et al. Silybin treatment is associated with reduction in serum ferritin in patients with chronic hepatitis C. J Clin Gastroenterol 2008;42:937-44.

[34] Federico A, Trappoliere M, Tuccillo C, et al. A new silybin-vitamin Ephospholipid complex improves insulin resistance and liver damage in patients with non-alcoholic fatty liver disease: preliminary observations. Gut 2006;55:901-2.

[35] Trappoliere M, Caligiuri A, Schmid M, et al. Silybin, a component of sylimarin, exerts anti-inflammatory and anti-fibrogenic effects on human hepatic stellate cells. J Hepatol 2009;50:1102-11.

[36] Caballero F, Fernandez A, Matias N, et al. Specific contribution of methionine and choline in nutritional nonalcoholic steatohepatitis: impact on mitochondrial S-adenosyl-1-methionine and glutathione. J Biol Chem 2010;285:18528-36.

[37] Wei Y, Rector R S, Thyfault J P, et al. Nonalcoholic fatty liver disease and mitochondrial dysfunction. World J Gastroenterol 2008;14:193-9.

[38] Rector R S, Thyfault J P, Uptergrove G M, et al. Mitochondrial dysfunction precedes insulin resistance and hepatic steatosis and contributes to the natural history of non-alcoholic fatty liver disease in an obese rodent model. J Hepatol 2010;52:727-36.

[39] Serviddio G, Bellanti F, Giudetti A M, et al. A silybin-phospholipid complex prevents mitochondrial dysfunction in a rodent model of nonalcoholic steatohepatitis. J Pharmacol Exp Ther 2010;332:922-32.

[40] Han D, Ybanez M D, Ahmadi S, et al. Redox regulation of tumor necrosis factor signaling. Antioxid Redox Signal 2009;11:2245-63.

[41] Khaper N, Bryan S, Dhingra S, et al. Targeting the vicious inflammationoxidative stress cycle for the management of heart failure. Antioxid Redox Signal 2010;13:1033-4.

[42] Garcia-Ruiz I, Rodriguez-Juan C, Diaz-Sanjuan T, et al. Uric acid and anti-TNF antibody improve mitochondrial dysfunction in ob/ob mice. Hepatology

[43] Petersen A M, Pedersen B K. The role of IL-6 in mediating the anti-inflammatory effects of exercise. J Physiol Pharmacol 2006;57 (Suppl. 10):43-51.

[44] Hong F, Radaeva S, Pan H N, et al. Interleukin 6 alleviates hepatic steatosis and ischemia/reperfusion injury in mice with fatty liver disease. Hepatology 2004;40:933-41.

[45] Sanchez-Garrido M A, Chico Y, Gonzalez R, et al. Interleukin-6 is associated with liver lipid homeostasis but not with cell death in experimental hepatic steatosis. Innate Immun 2009;15:337-49.

[46] Chabowski A, Zmijewska M, Górski J, et al. Effect of IL-6 deficiency on myocardial expression of fatty acid transporters and intracellular lipid deposits. J Physiol Pharmacol 2007;58:73-82.

[47] Kassel K M, Guo G L, Tawfik O, et al. Monocyte chemoattractant protein-1 deficiency does not affect steatosis or inflammation in livers of mice fed a methionine-choline-deficient diet. Lab Invest 2010;90:1794-804.

[48] Svegliati-Baroni G, Candelaresi C, Saccomanno S, et al. A model of insulin resistance and nonalcoholic steatohepatitis in rats: role of peroxisome proliferator-activated receptor-alpha and n-3 polyunsaturated.

1-12. (canceled)

13. A method for treating non-ischemic cardiomyopathy in an NAFLD patient comprising administering a therapeutically effective amount of silibinin to said patient.

14. The method according to claim 13, wherein before the treatment the patient has morphological abnormalities of the myocardiocytes.

15. The method according to claim 14, wherein after the treatment, the morphological abnormalities of the myocardiocytes of the patient are reversed and the myocardial morphology is similar to that of a healthy individual.

16. The method according to claim 14, wherein the morphological abnormalities of the myocardiocytes are selected from the group consisting of diffuse vacuolar degeneration, intracellular lipid accumulation, myocardiocyte abnormal size and altered nuclear morphology.

17. The method according to claim 14, wherein the morphological abnormalities of the myocardiocytes are selected from the group consisting of diffuse vacuolar degeneration, intracellular lipid accumulation, myocardiocyte abnormal size and altered nuclear morphology.

18. The method according to claim 13, wherein after the treatment, there is an antisteatotic effect of Silibinin in the heart.

19. The method according to claim 13, wherein the Silibinin is administered in a pharmaceutical formulation.

20. The method according to claim 13, wherein the Silibinin is administered via enteral or parenteral route.
